(19) United States
(12) Patent Application Publication
(10) Pub. No.: US 2024/0245545 A1
(43) Pub. Date: Jul. 25, 2024

(54) Overnight Urinal Device

(71) Applicant: Mary Helton, Toledo, IL (US)

(72) Inventor: Mary Helton, Toledo, IL (US)

(21) Appl. No.: 18/362,075

(22) Filed: Jul. 31, 2023

Related U.S. Application Data
(60) Provisional application No. 63/440,761, filed on Jan. 24, 2023.

(51) Int. Cl. A61F 5/453

(52) U.S. Cl. CPC A61F 5/453

(57) ABSTRACT

A novel urine-collecting device is disclosed. The device is a urine collection container with a large capacity to hold urine to reduce the need for frequent emptying and to prevent overflow during the night. The urine-collecting device comprises a body component comprised of a five-gallon container. Further, the device comprises a male external catheter which is attached to a tube that flows into the five-gallon container. The size of the container may vary, depending on the needs and/or wants of a user.

### CROSS-REFERENCE TO RELATED APPLICATION

The present application claims priority to, and the benefit of, U.S. Provisional Application No. 63/440,761, which was filed on Jan. 24, 2023, and is incorporated herein by reference in its entirety.

### FIELD OF THE INVENTION

The present invention relates generally to the field of urine-collecting devices. More specifically, the present invention relates to a large-capacity container for collecting urine from bedridden individuals. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in urine-collecting devices. Generally, conventional urine-collecting containers, such as bedpans, are limited in their capacity to hold urine. They have to be emptied often to ensure that they do not overflow. When caretakers or nursing staff are unavailable to empty the container, urine will overflow, saturating the bed and/or floor and requiring cleanup and sanitization.

In order to determine how the kidneys will adjust to changing physiologic needs over a long period, it can be standard to collect all urine produced for 24 hours. Since substances are excreted by the kidneys at different rates and amounts, in particular the concentration of catecholamine and its derivatives, during the day such a long-term collection of all urine generated over a 24-hour period provides a wealth of usable and normally fairly accurate information. Thus, to collect urine over a 24-hour period on an outpatient basis, the patient must have a large capacity urine-collecting device.

Accordingly, a need remains for a large capacity urine-collecting device that can be used for collecting urine from bedridden individuals. Further, a large capacity urine-collecting device is necessary to hold a large amount of urine and save valuable time for nursing staff.

Therefore, there exists a long-felt need in the art for a urine-collecting device that provides users with a large capacity container for collecting urine from bedridden individuals. There is also a long-felt need in the art for a urine-collecting device that holds a large amount of urine so that it does not overflow during the night. Further, there is a long-felt need in the art for a urine-collecting device that reduces the number of times the container needs to be emptied. Moreover, there is a long-felt need in the art for a device that saves valuable time for caretakers and nursing staff by holding more liquid than conventional containers. Further, there is a long-felt need in the art for a urine-collecting device that ensures individuals can urinate comfortably from their bed when needed. Finally, there is a long-felt need in the art for a urine-collecting device that utilizes a five-gallon container.

The subject matter disclosed and claimed herein, in one embodiment thereof, comprises a urine-collecting device. The device is a urine collection container with a large capacity to hold urine to reduce the need for frequent emptying and to prevent overflow during the night. The urine-collecting device comprises a body component comprised of a five-gallon container. Further, the device comprises a male external catheter which is attached to a tube that flows into the five-gallon container. The size of the container may vary, depending on the needs and/or wants of a user.

In this manner, the urine-collecting device of the present invention accomplishes all of the foregoing objectives and provides users with a device that provides a user with a large capacity collecting container for urine. The device is a five-gallon container that is secured to a catheter. The device reduces the need for frequent emptying and prevents overflow.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a urine-collecting device. The device is a urine collection container with a large capacity to hold urine to reduce the need for frequent emptying and to prevent overflow during the night. The urine-collecting device comprises a body component comprised of a five-gallon container. Further, the device comprises a catheter which is attached to a tube that flows into the five-gallon container.

Generally, urine is sometimes needed to be collected over a 24-hour period for bed-ridden patients, but conventional urine-collecting containers are not large enough and must be emptied frequently. Further, the frequent emptying of most urine-collecting containers puts strains on nursing staff and causes issues will urine overflow. The present invention discloses a device that provides for large-capacity urine collection, without the worry of overflow and/or messes on the floor or bed.

In one embodiment, the urine-collecting device comprises a body component. The body component can be any suitable shape and size as is known in the art. Generally, the body component is a cylindrical-shaped container with a bottom closed end and curved sides, as well as an open cavity that is accessed from an open end.

In one embodiment, the body component is approximately a five-gallon container, however, any other suitable-sized containers can be utilized as is known in the art. Specifically, the size of the container may vary, depending on the needs and/or wants of a user. In one embodiment, the body component is a one-gallon container.

In one embodiment, the sides of the body component comprise measurement gradient markings, to designate the volume of the body component. The measurement gradient markings can be positioned on either side or both sides of the body component, depending on the needs and/or wants of a user.

In one embodiment, the bottom of the body component comprises a plurality of wheels, which aid in the transportation of the device, especially after it is filled with urine. The plurality of wheels may be disposed on the underside of the body component and may allow the device to roll in any direction. In other words, the plurality of wheels may rotate, swivel, and pivot, based on a user's preferences. Specifically, the plurality of wheels may rotate, swivel, and pivot when the device is pushed and/or pulled. The plurality of wheels can be secured to an underside of the body component via any suitable securing means as is known in the art, such as bolts, screws, pins, etc. Further, any suitable wheels can be utilized as is known in the art, such as castors, rollers, swivel wheels, etc. In one embodiment, the plurality of wheels are rugged, all-terrain wheels that help the device travel across thick carpet or other rough flooring. Additionally, any suitable number of wheels can be utilized as is known in the art, depending on the needs and/or wants of a user.

In one embodiment, the body component comprises a removable screw cap component. The cap component allows the body component to be sealed to prevent unpleasant smells or accidental spills. The cap component is removable to allow the body component to be emptied, as needed and then resealed. The cap component can be screwed, snap fit, pin fit, latches, etc. Further, the cap component can comprise instructions for the user about what to do with the urine-collecting device and how to open and/or close the device, as well as how to connect the catheter.

In one embodiment, the body component comprises a handle that is secured to each side, allowing a user to easily transport the device, as needed. The handle would be secured via pins, screws, bolts, glue, etc., or any other suitable means as is known in the art.

In one embodiment, the cap of the body component comprises a through hole, allowing a flexible tubing to be inserted and secured. Specifically, the flexible tubing can be any suitable diameter and length, depending on the wants and/or needs of a user. The flexible tubing would comprise a first end and a second end. The through hole would comprise a hose barb that secures a first end of the flexible tubing to the through hole, or the flexible tubing can comprise a hose clamp that secures it to the through hole. The flexible tubing would be manufactured of any suitable elastomeric plastic, as is known in the art.

In one embodiment, the second end of the flexible tubing is secured to a catheter. The flexible tubing would be secured to the catheter via fittings, clamps, etc., or any other suitable fastening devices as is known in the art. The catheter can be any suitable catheter as is known in the art, including intermittent catheters, indwelling catheters, and external catheters. The catheter is then secured to a user via any suitable means as is known in the art (i.e., as a conventional catheter is secured to a user).

In one embodiment, the urine-collecting device is manufactured of a lightweight, durable material such as plastic or other polymers, or the like and manufactured through common molding processes. Specifically, the device can be manufactured from heat-sealable plastic or polymers, such as polypropylene or acrylonitrile-butadiene-styrene (ABS), or any other suitable material as is known in the art, such as, but not limited to, polycarbonate, polyethylene, polyethylene terephthalate, polyvinyl chloride, polystyrene, etc. Generally, the device is also manufactured from a waterproof material, or the body component comprises a waterproof coating. Furthermore, the device may be molded in transparent or translucent plastics, such that a user can view the interior of the body component, to see how full the device is. In another embodiment, the body component can be manufactured of antibacterial or antimicrobial material, or the body component comprises a coating that is antibacterial or antimicrobial. In yet another embodiment, the urine-collecting device comprises a plurality of indicia.

In yet another embodiment, a method of collecting urine without the need for frequent emptying is disclosed. The method includes the steps of providing a urine-collecting device comprising a body component with a tube attached to an external catheter. The method also comprises securing the external catheter to a user. Further, the method comprises securing an end of the external catheter to a flexible tubing. The method comprises securing the flexible tubing within the body component. Finally, the method comprises utilizing the external catheter and filling the body component without the need for frequent emptying.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the urine-collecting device of the present invention showing the catheter secured to a tube that flows into the container in accordance with the disclosed architecture;

FIG. 2 illustrates a top perspective view of one embodiment of the urine-collecting device of the present invention showing the urine-collecting device in use in accordance with the disclosed architecture; and

FIG. 3 illustrates a flowchart showing the method of collecting urine without the need for frequent emptying in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a long-felt need in the art for a urine-collecting device that provides users with a large capacity container for collecting urine from bedridden individuals. There is also a long-felt need in the art for a urine-collecting device that holds a large amount of urine so that it does not overflow during the night. Further, there is a long-felt need in the art for a urine-collecting device that reduces the number of times the container needs to be emptied. Moreover, there is a long-felt need in the art for a device that saves valuable time for caretakers and nursing staff by holding more liquid than conventional containers. Further, there is a long-felt need in the art for a urine-collecting device that ensures individuals can urinate comfortably from their bed when needed. Finally, there is a long-felt need in the art for a urine-collecting device that utilizes a five-gallon container.

The present invention, in one exemplary embodiment, is a novel urine-collecting device. The device is a urine collection container with a large capacity to hold urine to reduce the need for frequent emptying and to prevent overflow during the night. The urine-collecting device comprises a body component comprised of a five-gallon container. Further, the device comprises a male external catheter which is attached to a tube that flows into the five-gallon container. The present invention also includes a novel method of collecting urine without the need for frequent emptying. The method includes the steps of providing a urine-collecting device comprising a body component with a tube attached to an external catheter. The method also comprises securing the external catheter to a user. Further, the method comprises securing an end of the external catheter to a flexible tubing. The method comprises securing the flexible tubing within the body component. Finally, the method comprises utilizing the external catheter and filling the body component without the need for frequent emptying.

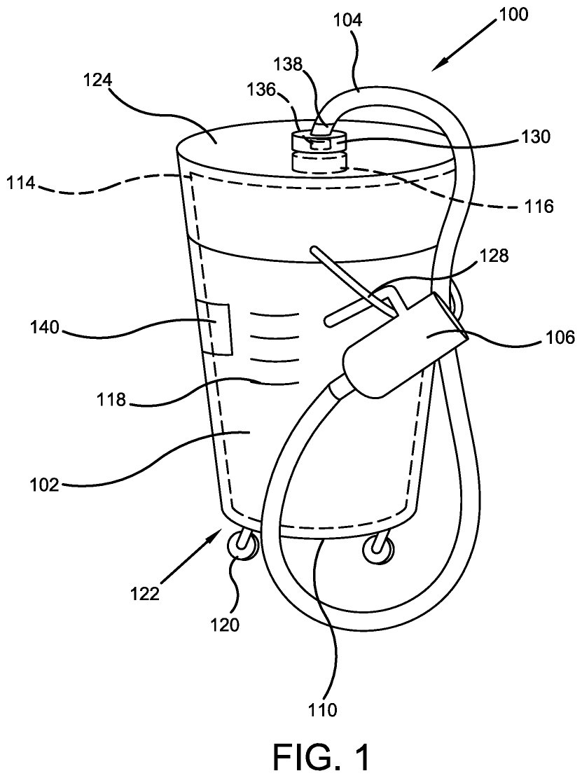

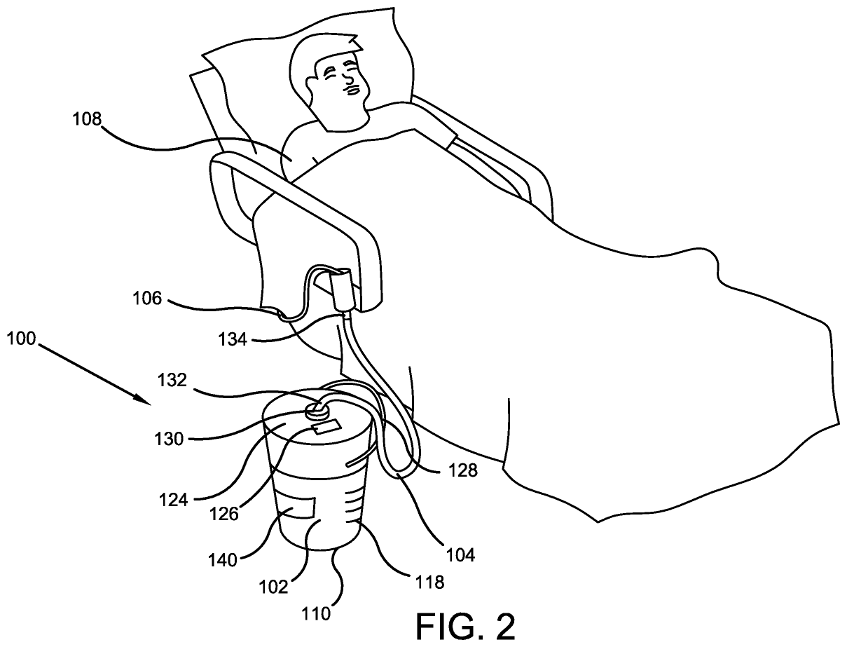

Referring initially to the drawings, FIGS. 1-2 illustrate a perspective view of one embodiment of the urine-collecting device 100 of the present invention. In the present embodiment, the urine-collecting device 100 is an improved urine-collecting device 100 that provides a user with a large capacity container for holding urine to reduce the need for frequent emptying and to prevent overflow during the night. Specifically, the urine-collecting device 100 comprises a body component 102 with a flexible tubing 104 and a catheter 106. The catheter 106 is secured to a user 108 and then attached to the flexible tubing 104. The flexible tubing 104 is then secured to the body component 102, which allows the flexible tubing 104 to empty into the body component 102, during use.

Generally, there are multiple forms of urinary catheters 106, such as intermittent, indwelling, and external catheters. All types of catheters 106 can be utilized with the urine-collecting device 100. If the catheter 106 is an intermittent catheter or indwelling catheter, a hollow tube is inserted into the bladder to drain urine. Intermittent catheters are used to treat urinary retention, and indwelling catheters are used to treat urinary retention and urinary incontinence. Intermittent catheters are catheters that are inserted into the bladder for a short duration to empty the bladder of urine, several times throughout the day. Indwelling catheters are inserted into the bladder and made to remain there to provide continuous urinary drainage. The external catheter does not enter the body. It has a condom-shaped receptacle that wraps around a man's penis and a thin tube that leads from the receptacle into a urine collection device. It does not treat urinary retention, only urinary incontinence. Many men find this style to be a great alternative to the more invasive catheters that require insertion through the urethra.

Typically, urine is sometimes needed to be collected over a 24-hour period for bed-ridden patients, but conventional urine-collecting containers are not large enough and must be emptied frequently. Further, the frequent emptying of most urine-collecting containers puts strains on nursing staff and causes issues will urine overflow. The present invention discloses a device 100 which provides for large-capacity urine collection, without worry of overflow and/or messes on the floor or bed.

Generally, the urine-collecting device 100 comprises a body component 102. The body component 102 can be any suitable shape and size as is known in the art. Generally, the body component 102 is a cylindrical-shaped container with a bottom closed end 110 and curved sides 112, as well as an open cavity 114 that is accessed from an open end 116. Thus, the body component 102 is generally shaped as a bucket or a pail but can be any suitable shape as needed, depending on the needs and/or wants of a user 108.

Furthermore, the body component 102 is approximately a five-gallon container, however, any other suitable sized containers can be utilized as is known in the art. Specifically, the size of the container may vary, depending on the needs and/or wants of a user. In one embodiment, the body component 102 is a one-gallon container, in other embodiments, the body component 102 is a three-gallon container, etc.

In one embodiment, the curved sides 112 of the body component 102 comprise measurement gradient markings 118, to designate the volume of the contents of the body component 102. The measurement gradient markings 118 can be positioned on either side 112 or both sides 112 of the body component 102, depending on the needs and/or wants of a user 108 and help a user 108 determine how much urine is within the body component 102.

Additionally, in another embodiment, the bottom 110 of the body component 102 comprises a plurality of wheels 120, which aid in the transportation of the device 100, especially after it is filled with urine. The plurality of wheels 120 may be disposed on the underside 122 of the body component 102 and may allow the device 100 to roll in any direction. In other words, the plurality of wheels 120 may rotate, swivel, and pivot, based on a user's preferences. Specifically, the plurality of wheels 120 may rotate, swivel, and pivot when the device 100 is pushed and/or pulled. The plurality of wheels 120 can be secured to an underside 122 of the body component 102 via any suitable securing means as is known in the art, such as bolts, screws, pins, etc. Further, any suitable wheels 120 can be utilized as is known in the art, such as castors, rollers, swivel wheels, etc. In one embodiment, the plurality of wheels 120 are rugged, all-terrain wheels that help the device 100 travel across thick carpet or other rough flooring. Additionally, any suitable number of wheels 120 can be utilized as is known in the art, depending on the needs and/or wants of a user 108.

In yet another embodiment, the body component 102 comprises a removable screw cap component 124. The cap component 124 allows the body component 102 to be sealed to prevent unpleasant smells or accidental spills. The cap component 124 is removable to allow the body component 102 to be emptied and cleaned, as needed and then resealed. The cap component 124 can be screwed, snap fit, pin fit, latches, etc., onto the body component 102. Further, the cap component 124 can comprise printed instructions 126 for the user 108 about what to do with the urine-collecting device 100 and how to open and/or close the device 100, as well as how to connect the catheter 106, or any other suitable instructions as is known in the art.

Further, the body component 102 comprises a handle 128 that is secured to each side 112, allowing a user 108 to easily transport the device 100, as needed. The handle 128 would be secured via pins, screws, bolts, glue, etc., or any other suitable means as is known in the art. Further, the handle 128 can be any suitable shape and size as is known in the art, as long as it can act to help in transporting the device 100.

Additionally, the cap 124 of the body component 102 comprises a through hole 130, allowing a flexible tubing 104 to be inserted and secured. Specifically, the flexible tubing 104 can be any suitable diameter and length, depending on the wants and/or needs of a user 108. The flexible tubing 104 would comprise a first end 132 and a second end 134. The through hole 130 would comprise a hose barb 136 that secures a first end 132 of the flexible tubing 104 to the through hole 130, or the flexible tubing 104 can comprise a hose clamp 138 which secures it to the through hole 130. The flexible tubing 104 would be manufactured of any suitable elastomeric plastic, as is known in the art.

Generally, the second end 134 of the flexible tubing 104 is secured to a catheter 106. The flexible tubing 104 would be secured to the catheter 106 via fittings, clamps, etc., or any other suitable fastening devices as is known in the art. The catheter 106 can be any suitable catheter as is known in the art, including intermittent catheters, indwelling catheters, and external catheters. The catheter 106 is then secured to a user 108 via any suitable means as is known in the art (i.e., generally, as a conventional catheter 106 is secured to a user 108).

Typically, the urine-collecting device 100 is manufactured of a lightweight, durable material such as plastic or other polymers, or the like and manufactured through common molding processes. Specifically, the device 100 can be manufactured from heat-sealable plastic or polymers, such as polypropylene or acrylonitrile-butadiene-styrene (ABS), or any other suitable material as is known in the art, such as, but not limited to, polycarbonate, polyethylene, polyethylene terephthalate, polyvinyl chloride, polystyrene, etc. Generally, the device 100 is also manufactured from a waterproof material, or the body component 102 comprises a waterproof coating. Furthermore, the device 100 may be molded in transparent or translucent plastics, such that a user 108 can view the interior (i.e., cavity 114) of the body component 102, to see how full the device 100 is. In another embodiment, the body component 102 can be manufactured of antibacterial or antimicrobial material or the body component 102 comprises a coating that is antibacterial or antimicrobial.

In yet another embodiment, the urine-collecting device 100 comprises a plurality of indicia 140. The body component 102 of the device 100 may include advertising, a trademark, or other letters, designs, or characters, printed, painted, stamped, or integrated into the body component 102, or any other indicia 140 as is known in the art. Specifically, any suitable indicia 140 as is known in the art can be included, such as, but not limited to, patterns, logos, emblems, images, symbols, designs, letters, words, characters, animals, advertisements, brands, etc., that may or may not be urine collection, or brand related.

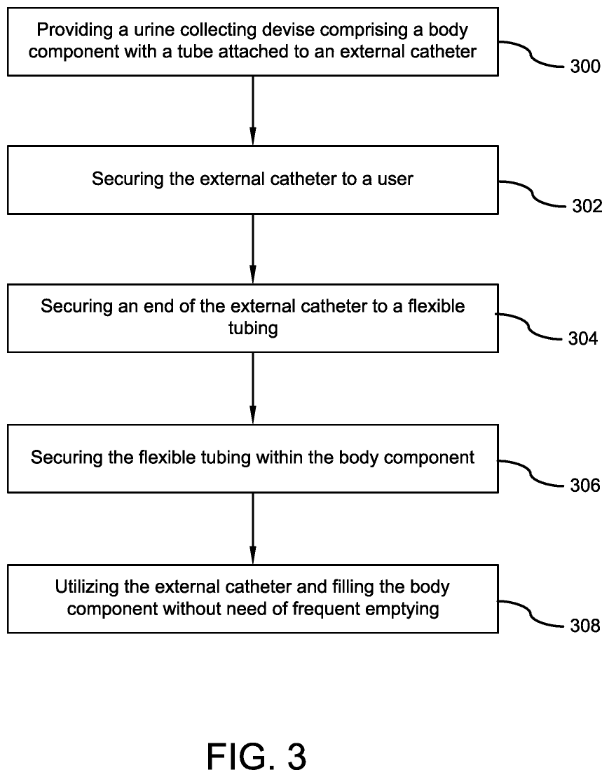

FIG. 3 illustrates a flowchart of the method of collecting urine without the need for frequent emptying. The method includes the steps of at 300, providing a urine-collecting device comprising a body component with a tube attached to an external catheter. The method also comprises at 302, securing the external catheter to a user. Further, the method comprises at 304, securing an end of the external catheter to a flexible tubing. The method comprises at 306, securing the flexible tubing within the body component. Finally, the method comprises at 308, utilizing the external catheter and filling the body component without the need for frequent emptying.

Certain terms are used throughout the following description and claims to refer to particular features or components. As one skilled in the art will appreciate, different users, may refer to the same feature or component by different names. This document does not intend to distinguish between components or features that differ in name but not structure or function. As used herein “ urine-collecting device”, “collecting device”, and “device” are interchangeable and refer to the urine-collecting device 100 of the present invention.

Notwithstanding the foregoing, the urine-collecting device 100 of the present invention can be of any suitable size and configuration as is known in the art without affecting the overall concept of the invention, provided that it accomplishes the above-stated objectives. One of ordinary skill in the art will appreciate that the urine-collecting device 100 as shown in FIGS. 1-3 are for illustrative purposes only, and that many other sizes and shapes of the urine-collecting device 100 are well within the scope of the present disclosure. Although the dimensions of the urine-collecting device 100 are important design parameters for user convenience, the urine-collecting device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A urine-collecting device that provides a user with a large capacity container for holding urine, the urine-collecting device comprising:
a body component that comprises approximately a five-gallon volume;
a flexible tubing; and
a catheter;
wherein the catheter is secured to a user and then attached to the flexible tubing;
wherein the flexible tubing is then secured to the body component; and
further wherein the flexible tubing empties urine into the body component, during use.

2. The urine-collecting device of claim 1, wherein the body component is a cylindrical-shaped container with a bottom closed end, curved sides, an open end, and an open cavity that is accessed from the open end.

3. The urine-collecting device of claim 2, wherein the curved sides of the body component comprise measurement gradient markings, to designate a volume of contents of the body component.

4. The urine-collecting device of claim 3, wherein the body component comprises a plurality of wheels for transportation, which are disposed on an underside of the body component.

5. The urine-collecting device of claim 4, wherein the body component comprises a removable cap component, that seals the body component to prevent unpleasant smells or accidental spills.

6. The urine-collecting device of claim 5, wherein the removable cap component is screwed onto the body component.

7. The urine-collecting device of claim 6, wherein the removable cap component comprises printed instructions for the user about what to do and how to operate the urine-collecting device.

8. The urine-collecting device of claim 7, wherein the body component comprises a handle that is secured to the curved sides, allowing a user to easily transport the urine-collecting device.

9. The urine-collecting device of claim 8, wherein the removable cap component comprises a through hole, allowing the flexible tubing to be inserted and secured.

10. The urine-collecting device of claim 9, wherein the flexible tubing comprises a first end and a second end, and the through hole comprises a hose barb that secures the first end to the through hole.

11. The urine-collecting device of claim 10, wherein the flexible tubing comprises a hose clamp which secures the first end of the flexible tubing through the hole.

12. The urine-collecting device of claim 10, wherein the second end of the flexible tubing is secured to the catheter, which is then secured to the user.

13. A urine-collecting device that provides a user with a large capacity container for holding urine, the urine-collecting device comprising:
a body component that comprises approximately a five-gallon volume;
a flexible tubing comprising a first end and a second end; and
a catheter; and
wherein the body component is a cylindrical-shaped container with a bottom closed end, curved sides, an open end, and an open cavity that is accessed from the open end;
wherein the curved sides of the body component comprise measurement gradient markings, to designate a volume of contents of the body component;
wherein the body component comprises a removable cap component, that seals the body component to prevent unpleasant smells or accidental spills;
wherein the body component comprises a handle that is secured to the curved sides, allowing a user to easily transport the urine-collecting device;
wherein the removable cap component comprises a through hole with a hose barb, allowing the first end of the flexible tubing to be inserted and secured;
wherein the second end of the flexible tubing is secured to the catheter, which is then secured to the user; and
further wherein the flexible tubing empties urine into the body component via the through hole, during use.

14. The urine-collecting device of claim 13 further comprising a plurality of indicia.

15. The urine-collecting device of claim 13, wherein the body component comprises a plurality of wheels for transportation.

16. The urine-collecting device of claim 15, wherein the plurality of wheels are disposed on an underside of the body component via screws.

17. The urine-collecting device of claim 13, wherein the removable cap component is screwed onto the body component.

18. The urine-collecting device of claim 13, wherein the removable cap component comprises printed instructions for the user about what to do and how to operate the urine-collecting device.

19. The urine-collecting device of claim 13, wherein the flexible tubing comprises a hose clamp which secures the first end of the flexible tubing through the hole.

20. A method of collecting urine without the need for frequent emptying, the method comprising the following steps:
providing a urine-collecting device comprising a body component with a tube attached to an external catheter;
securing the external catheter to a user;
securing an end of the external catheter to a flexible tubing;
securing the flexible tubing within the body component; and
utilizing the external catheter and filling the body component without the need for frequent emptying.
